(19) United States
(12) Patent Application Publication
(10) Pub. No.: US 2020/0170710 A1
(43) Pub. Date: Jun. 4, 2020

(54) SURGICAL DECISION SUPPORT USING A DECISION THEORETIC MODEL

(71) Applicant: THE GENERAL HOSPITAL CORPORATION, Boston, MA (US)

(72) Inventors: Daniela Rus, Weston, MA (US); Ozanan Meireles, Boston, MA (US); Guy Rosman, Newton, MA (US); Daniel Hashimoto, Cambridge, MA (US)

(21) Appl. No.: 16/638,270

(22) PCT Filed: Aug. 23, 2018
(86) PCT No.: PCT/US2018/047679

§ 371 Date: Feb. 11, 2020

Related U.S. Application Data
(60) Provisional application No. 62/549,272, filed on Aug. 23, 2017.

(51) Int. Cl. A61B 34/10; A61B 34/00; G16H 20/40; G16H 70/20; G06N 3/02

(52) U.S. Cl. CPC A61B 34/10; A61B 34/25; G16H 20/40; A61B 2562/02; G06N 3/02; A61B 2034/252; A61B 2034/107; G16H 70/20

(57) ABSTRACT

A surgical procedure on a patient is monitored at a sensor to provide an observation. A current surgical state is estimated as a belief state over of a plurality of surgical states, representing different phases of the surgery, from the observation and an observation function for each surgical state. A world state of a plurality of world states representing a state of one of the patient, a medical professional performing the surgical procedure, and the environment in which the surgical procedure is being conducted is estimated from the estimated surgical state. From the estimated surgical state, the estimated world state, and a model, at least one surgical state that will be entered during the surgical procedure is predicted and an output representing the predicted at least one surgical state is provided O at an associated output device.

### RELATED APPLICATIONS

The present application claims priority to U.S. Provisional Patent Application Ser. No. 62/549,272 filed Aug. 23, 2017 entitled SURGICAL DECISION THEORETIC ANALYSIS under Attorney Docket Number MGH 24623. The entire content of this application is incorporated herein by reference in its entirety for all purposes.

### TECHNICAL FIELD

This disclosure relates to systems and methods for decision support and, in particular, is directed to systems and methods for surgical decision support using a decision theoretic model.

### BACKGROUND

As surgical care quality increases with new technologies and greater understanding of surgical disease, gaps remain in both access to and quality of care for many patients. This has led to minimal volume pledges that restrict surgical procedures to surgeons and hospitals with an arbitrarily determined number of sufficient annual cases. Volume pledges have raised concerns over the potential regionalization of surgical care and the impact that regionalization may have on access to surgery, particularly for rural areas. High volume hospitals for complex operations are not readily accessible to many patients, and recent work has shown, for example, that rural patients with cancer are more likely to have their resections performed at a low-volume, yet local, hospital. There is also evidence to suggest that regionalization of care would disproportionately affect minorities and patients without private insurance, as they are most likely to have their operations performed at low-volume hospitals. Thus, the proposed redistribution of care with volume pledges may not be the best solution for all patients.

An estimated 234.2 million operations are performed annually worldwide, but surgeons learn from one patient at a time, limiting their knowledge on rate procedures. Residency is designed to give surgeons the fundamental skills necessary to apply and expand principles of safe surgery to each situation encountered in practice, even novel situations. However, residency relies on apprenticeship-like exposure to experienced surgeons. These experienced surgeons, with a wealth of experiential data, have limited availability. Training for rare cases has thus necessarily been left to a limited number of surgeons who complete sub-specialty fellowships which are often housed in high volume, urban academic centers, again leaving rural and minority populations with a disadvantage in access to care.

Previous attempts have been made to accumulate and distribute intraoperative decision-making models to surgeons to optimize surgical care. Cognitive task analysis (CTA) has been used to codify and distill experienced surgeons' knowledge into standardized checklists to assist in decision-making. In surgical patients, up to 67% of errors occur intraoperatively, and of those errors, 86% of errors are secondary to cognitive factors such as failures in judgment or memory that lead to poor decisions. However, CTA is limited by the fact that 50-75% of decisions made in surgery can be lacking in the conscious recall of surgeons due to either inexperience or automaticity, and these efforts have been time consuming and have not addressed morbidity and mortality at a large scale.

### SUMMARY

In accordance with an aspect of the present invention, a system is provided. At least one sensor is positioned to monitor a surgical procedure on a patient. A processor is operatively connected to a non-transitory computer that stores machine executable instructions for providing a surgical decision support system, such that the machine executable instructions are executed by the processor to provide each of a sensor interface that receives data from the at least one sensor and generates observations from the received data, a surgical model, an agent, and a user interface.

The surgical model includes a plurality of surgical states, each representing different phases of the surgery, an observation function for each surgical state representing at least one likelihood of a given observation from the sensor interface given the surgical state, a plurality of actions that can be taken by a surgeon to transition between states of the plurality of surgical states, a plurality of world states, each representing a state of one of the patient and the environment in which the surgical procedure is being conducted, a set of effectors, each representing a likelihood of a transition between a given world state and another world state given a specific surgical state, a set of transition probabilities, each representing a likelihood of a transition from a given surgical state to another surgical state given each of a specific world state and a selected action of the plurality of actions, and a rewards function defining respective reward values for each of at least two ordered pairs. Each of the at least two ordered pairs representing a surgical state of the plurality of surgical states and a world state of the plurality of world states.

The agent estimates current surgical state and world state distributions as a belief state and selects at least one of the plurality of actions as to optimize an expected reward given at least one observation from the sensor interface. The user interface provides one of the selected at least one of the plurality of actions, a likelihood that a selected surgical state will be entered in the course of the procedure, and an expected final world state to an associated output device. The output device provides the one of the selected at least one of the plurality of actions, the likelihood that the selected surgical state will be entered in the course of the procedure, and an expected final world state to a user in a form comprehensible by a human being.

In accordance with another aspect of the present invention, a method is provided. A surgical procedure on a patient is monitored at a sensor to provide an observation. A current surgical state is estimated as a belief state over of a plurality of surgical states, representing different phases of the surgery, from the observation and an observation function for each surgical state. A world state of a plurality of world states representing a state of one of the patient and the environment in which the surgical procedure is being conducted is estimated from the estimated surgical state. From the estimated surgical state, the estimated world state, and a model, at least one surgical state that will be entered during the surgical procedure is predicted and an output representing the predicted at least one surgical state is provided at an associated output device.

In accordance with yet another aspect of the present invention, a method is provided. A plurality of surgical procedures are monitored at a sensor to provide a plurality of time series of observations. For each of a plurality of surgical states representing different phases of a surgical procedure, an observation function and a set of transition probabilities are learned from the plurality of time series of observations. Each observation function represents at least one likelihood of a given observation from the sensor given a surgical state. Each set of transition probabilities represents a likelihood of a transition from a given surgical state to another surgical state given each of a specific world state of a plurality of world states and a selected action of a plurality of actions. A set of effectors are learned from the plurality of time series of observations. Each set of effectors represents a likelihood of a transition between a given world state of the plurality of world states and another world state of the plurality of world states given a specific surgical state. An associated rewards function is generated defining respective reward values for each of at least two ordered pairs. Each of the at least two ordered pairs representing a surgical state of the plurality of surgical states and a world state of the plurality of world states.

### BRIEF DESCRIPTION OF THE DRAWINGS

FIG. 1 illustrates an example of a system for surgical decision support;

FIG. 2 illustrates a portion of one example of a model that might be used in the system of FIG. 1;

FIG. 3 illustrates a method for assisting surgical decision making using a model trained via reinforcement learning;

FIG. 4 illustrates a method for providing a surgical model, for example, for an assisted surgical decision making method like that presented in FIG. 3; and

FIG. 5 illustrates a computer system that can be employed to implement systems and methods described herein.

### DETAILED DESCRIPTION

The systems and methods presented herein seek to instead boost the effective experience of surgeons by data mining operative sensor data, such as video, to generate a collective surgical experience that can be utilized to provide automated predictive-assistive tools for surgery. Rapid advancements in streaming data analysis have opened the door to efficiently gather, analyze, and distribute collective surgical knowledge. However, simply collecting massive amounts of data is insufficient, and human analysis at the individual case level is costly and time-consuming. Therefore, any real solution must automatically summarize many examples to reason about rare (yet consequential) events that occur in surgery. The systems and methods presented herein provide a surgical decision-theoretic model (SDTM) that utilizes decision-theoretic tools in artificial intelligence (AI) to quantify qualitative knowledge of surgical decision making to allow for accurate, real-time, automated decision analysis and prediction.

AI has been used to describe or segment mostly linear sequences of events in surgery video analysis, but intraoperative decisions do not always follow a linear process, especially in emergency surgery or during unexpected events in elective surgery. The inventors have found that computational tools currently in clinical use lack the ability to analyze highly branched decision processes affected by many relevant factors, especially the patient state (e.g., inflammation, aberrant anatomy, etc.). By modeling surgery states and surgeon internal rewards over a large pool of operative videos as a decision theoretic model, the systems and methods presented herein can capture the value judgments made by the surgeon. Learning the SDTM over the dataset will thus evaluate the possible decision paths that can occur in a given operation in terms of causes and consequences. For example, if the critical view in a cholecystectomy is not achievable, a CBD injury can be avoided by performing a cholangiogram to assess biliary anatomy instead of clipping or cutting a presumed duct.

The proposed model provides a two-pronged approach to reducing the disparities in surgical care. First, surgical knowledge is collected from operative video of many different surgeons via automated processes to learn surgical techniques and decisions and disseminate this knowledge. This allows for automated analysis of key decision points in an operation and provides real-time feedback/guidance to surgeons that is augmented by predictive error recognition to improve surgical performance. By pooling the experience of multiple surgeons, SDTM could bring the decision-making capabilities of the collective surgical community into every operation. It will lay the groundwork for computer-augmented intraoperative decision-making with the potential to reduce or even eliminate patient morbidity and mortality caused by intraoperative performance. By equipping surgeons with automated decision-support tools for both training and intraoperative performance, we can target the operating room as an intervention to improve the quality of care being delivered to all populations.

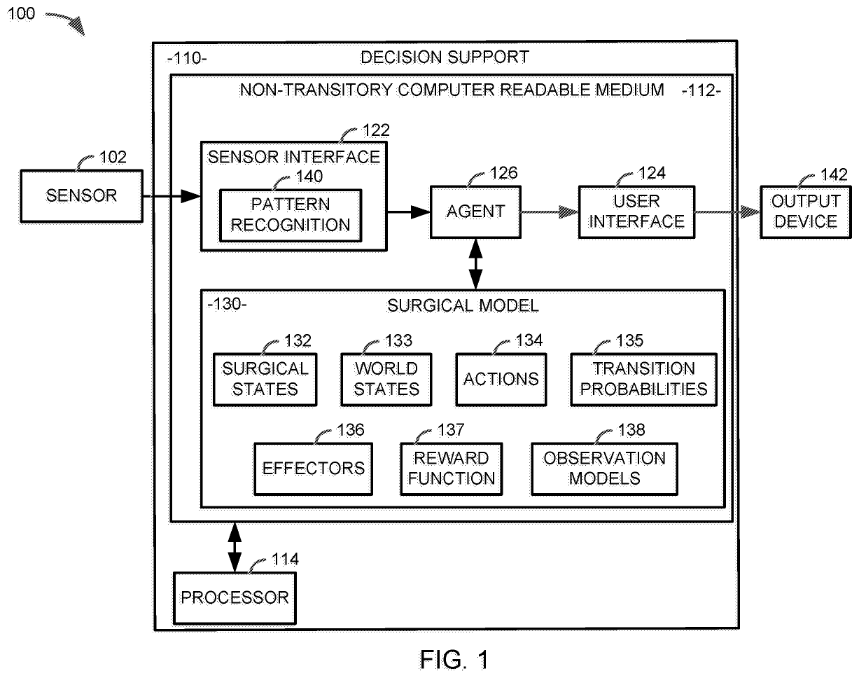

FIG. 1 illustrates an example of a system 100 for surgical decision support. The system 100 includes at least one sensor 102 positioned to monitor a surgical procedure on a patient. Sensors, for this purpose, can include video cameras, in the visible or infrared range, a microphone or other input device to receive comments from the surgical team at various time points within the surgery, accelerometers or radio frequency identification (RFID) devices disposed on a surgeon or an instrument associated with the surgical procedure, intraoperative imaging technologies, such as optical coherence tomography, computed tomography, X-ray imaging, sensor readings from other systems utilized in the surgical procedure, such as an anesthesia system, and sensors that detect biometric parameters of the patient, such as sphygmomanometers, in vivo pressure sensors, pulse oximeters, and electrocardiographs. The sensor data is provided to a decision support assembly 110. In the illustrated example, the decision support assembly 110 is implemented as machine executable instructions stored on a non-transitory computer readable medium 112 and executed by an associated processor 114. It will be appreciated, however, that the decision support assembly 110 could instead be implemented as dedicated hardware or programmable logic, or that the non-transitory computer readable medium 112 could comprise multiple, operatively connected, non-transitory computer readable media that are each either connected locally to the processor 114 or connected via a network connection.

The executable instructions stored on the non-transitory computer readable medium 112 include a sensor interface 122 that receives and conditions data from the at least one sensor 102, a user interface 124, an agent 126, and a model 130. The model 130 represents the surgical procedure as a progression through a first set of states 132, referred to herein as “surgical states.” The set of surgical states 132 can either be selected in advance, for example, by a human expert or learned as a non-parametric inference during training of the model 130. The model additionally represents the state of the patient and the environment as a second set of states 133, referred to herein as “world states.” The world states 133 can include a patient state description (e.g. inflammation, bleeding, aberrant anatomy, etc.) as well additional information about the patient, the medical professions performing the surgical procedure, including the surgeon, and the environment in which the surgical procedure is performed. Surgical states are linked by a set of actions 134 representing actions that can be taken by the surgeon. Specifically, in the model, a surgeon can take an action to transition, with a given transition probability from a set of learned transition probabilities 135, from one surgical state to another surgical state.

Entering a given surgical state can have an effect on the world state of the system, which is represented in the model 130 by a set of effectors 136 defining the interaction between these states probabilistically. The transition probabilities 135 governing transitions between surgical state for specific actions and the effectors 136 representing transitions between world states for specific surgical states can be determined from data generated in previous surgeries. Each world state and surgical state combination can be mapped to a particular reward via a reward function 137, reflecting how desirable it is, given our data from previous surgeries, for the surgical procedure to be in that combination of states. The reward function 137 maps the current patient state and surgery state into a reward, with negative rewards for complications or incomplete operations and positive rewards for a successful completion of the operation. Finally, each of the set of surgery states 132 is represented by an associated observation model from a set of observation models 138 representing the likelihood that an observation will be received from the sensor 102 given a particular surgical state.

The agent 126 estimates the current surgical state and world state from observations provided by one or more sensors associated with the system. It will be appreciated that the estimation of the current states is probabilistic, and thus the current state is estimated as a belief state, representing, for each of the plurality of surgical states, the likelihood that the surgical procedure is the surgical state. In one example, the sensor interface 122 can be include a discriminative pattern recognition system 140, such as a support vector machine or an artificial neural network (e.g., recurrent neural networks, such as long short-term memory and gated recurrent units), convolutional neural networks, and capsule networks), that generates an observation from the sensor data. The output of the discriminative pattern recognition system 140 can be provided to the agent 126 as an observation.

Once the belief state representing the distribution of surgical states has been established, the agent can then predict what surgical and world states will be entered during the surgery by determining a sequence of actions that will provide the maximum reward. In modeling these actions, it is assumed that the surgeon has perfect knowledge of the surgical state—the surgeon knows what actions that he or she has performed—and incomplete knowledge of the patient state. In fact, mistaken estimation of the patient state, as reflected in the world states, can lead to errors in the surgical procedure. In one example, transitions between states are modelled via a reinforcement learning process in a manner analogous to a hidden Markov Decision Process (hMDP) guided by the reward function. It will be appreciated, however, that the model of transitions among the surgical states are not a true Markov Decision Process as the transition probabilities among surgical states depend on the world state, not simply the current surgical state. Alternatively, an inverse reinforcement learning process or an imitation learning process can be used to generate the model. In another example, the model can be implemented with a recurrent neural network, for example, long short-term memory and gated recurrent units, representing the surgical state transitions conditioned to world states and the world state transitions probabilities, with or without conditioning on the sensor data.

In one example, in which the sensor 102 is a surgical video system, analysis of the surgery video involves estimating the surgery and patient state using the effectors 136 that relate the two. Explicit handling of patient state and surgeon state subdivides the problem into smaller, more manageable learning problems, avoiding the curse of dimensionality often encountered in large-scale machine learning problems. The unknown patient state lends itself to sampling due to its causal structure, and Markov chain Monte Carlo based approaches can be adapted for learning decision-making on the model. The hybrid structure, using both the surgical states 132 and the world states 133 is particularly useful, as many patient states are not directly observed for most of the video.

The agent 126 navigates the surgical model to select at least one of the plurality of actions as to optimize an expected reward given at least one observation from the sensor interface. Accordingly, from the model 130 and the current surgical and world states, the agent 126 can predict the log-probability that an observation, o, will be received during the surgical procedure from a sum of the surgeon's perceived reward and the log-probability of seeing the observation given the surgical states traversed over time such that the log-probability that an observation, o, will be received during the surgical procedure can be written as

∫ₜ[λ₁Σ_{t′}[γ^{t′}R(S(t+t′),A(t+t′))]+log P(S(t),S(t+1))+λ₂ log P(O(s)|Sₜ,Wₜ(],

wherein s is a member of the set of surgery states, S, 132, representing different phases of the surgery, A is the set of transitions, R is the rewards function 137, W is the set of world states 133, γ^{t′} is a discount rate applied to the reward function, and λ₁ and λ₂ are weighting factors. The term under summation with respect to t′ describes the total expected reward for the agent over future trajectories, with discount, γ^{t′}, as captured for a soft rational agent.

The agent 126 can predict the likelihood that the surgical procedure will enter a given state, for example, surgical state associated with a successful or unsuccessful procedure, given the current world state and surgical state. Accordingly, a likelihood that the surgical procedure will end in success or failure, given the current surgical and world states, can be maintained in real time, allowing a surgeon or member of the operating staff to be notified if the probability of success, given the current model of the surgeon's actions, falls below a threshold value. Similarly, the likelihood of entering one or more states associated with a given complication or resource use can be determined, and thus the likelihood of the complication arising or the resource being used can be estimated. Further, for a given surgical state and world state, the agent 126 can determine which action is likely to produce the best reward, and appropriate guidance can be provided to the surgeon in response to this determination.

The user interface 124 communicates predictions generated by the agent to human being via an appropriate output device 142, such as a video monitor, speaker, or network interface. The predictions can include, for example, a selected action of the plurality of actions, a likelihood that a selected surgical state will be entered in the course of the procedure, and an expected final world state to an associated output device. It will be appreciated that the predictions can be provided directly to the surgeon to guide surgical decision making. For example, if a complication or other negative outcome is anticipated without additional radiological imaging, the surgeon could be advised to wait until the appropriate imaging can be obtained.

In one implementation, the various surgical states 132 and world states 133 can be associated with corresponding resources. For example, if the agent 126 determines that a surgical state 132 representing a need for radiological imaging will be entered at some point in the surgery, the user interface 124 could transmit a message to a member of the operating team or another individual at the facility in which the surgical procedure is performed to request the necessary equipment. Similarly, if the agent predicts a progression through the surgical states that diverges from an expected progression, the user interface 124 could transmit a message to a coordinator for the facility in which the surgical procedure is performed to schedule additional time in the operating room. Accordingly, the system 100 can be used to not only to assist less experienced surgeons in less common surgical procedures or unusual presentations of more common surgical procedures, but to more efficiently allocate resources across a surgical facility.

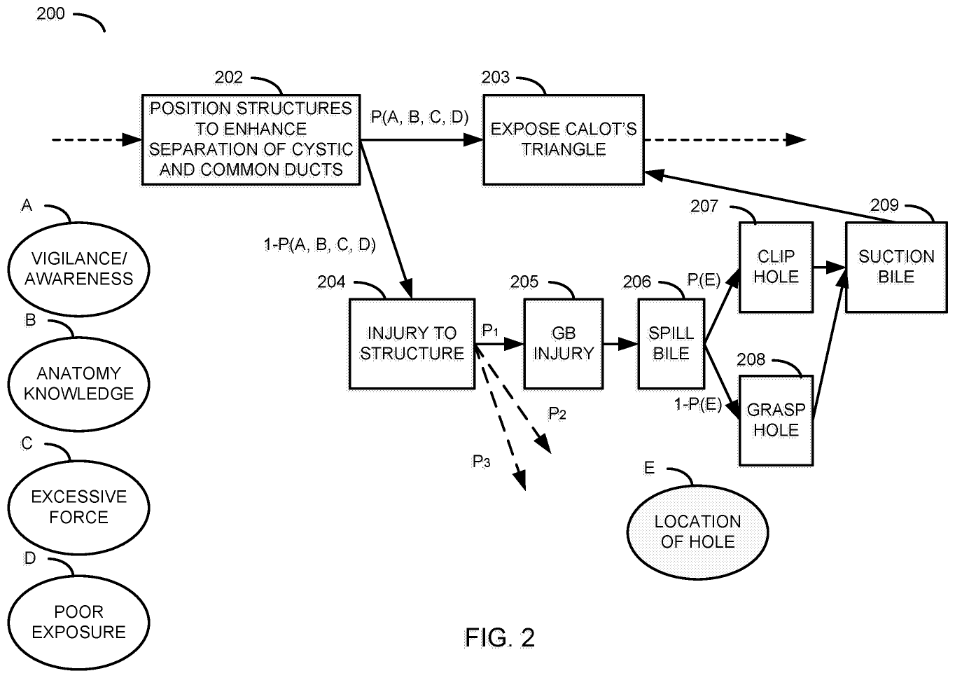

FIG. 2 illustrates a portion of one example of a model 200 that might be used in the system of FIG. 1. The illustrated portion of the model 200 includes a plurality of surgical states 202-209 and a plurality of world states A-E. World states A-D each represent an attribute of a surgeon performing the surgical procedure that can be determined from actions taken by the surgeon, or more specifically, surgical states entered during the procedure, at earlier stages of the procedure. In the illustrated example, the model 200 is intended for a laparoscopic cholecystectomy, although it will be appreciated that the general principles can be generalized to various surgical procedures.

In the example, a first world state A indicates that the surgeon has acted with a threshold level of vigilance during the procedure, a second world state B represents whether the surgeon has demonstrated a threshold level of anatomical knowledge in prior surgical states, a third world state C indicates whether the force applied by the surgeon in previous stages of the surgery was excessive, and a fourth world state D indicates poor exposure of the anatomy of interest, generally due to inexperience or lack of proficiency in laparoscopy. These world states A-D are binary, but it will be appreciated that the model will estimate the presence or absence of a given world state probabilistically, as neither the model nor the surgeon themselves have perfect knowledge of these states.

From the first illustrated surgical state 202, in which structures are positioned to enhance separation of cystic and common ducts, the surgeon can complete the action associated with this stage of the surgery, specifically, the positioning of the structures, to advance to a second surgical state 203. It will be appreciated, however, that this action will only advance the surgery without complication if properly performed, and thus the likelihood of advancing to the second surgical state 203 given the action is a probability, P. The specific probability of properly concluding the action is a function of the attributes of the surgeon described above, and thus the probability is actually a function of the world states, P(A, B, C, D). Similarly, the likelihood that the action fails to advance the surgery to the next surgical state without complication is 1-P(A, B, C, D).

With probability 1-P(A, B, C, D), the action taken at the first surgical state 202 leads to a third surgical state 204, representing an injury to an anatomical structure. While multiple injuries are possible, each presented by a probability, Pᵢ, the illustrated portion of the model contains only a gall bladder (GB) injury at a fourth surgical stage 205. As a result of this injury, bile will be spilled at a fifth surgical stage 206. At this point, the surgeon can take an action to clip the hole, at a sixth surgical stage 207 or grasp the hole at a seventh surgical stage 208. In modelling this decision by the surgeon, a fifth world state E becomes relevant, representing the location of the hole in the gall bladder. Unlike the first four world states A-D, world state E models a state of a patient, and is shown with shading to represent this difference. It will be appreciated, however, that world states are treated similarly by the model regardless of the underlying portion of the surgical environment that they represent. Accordingly, the model predicts that the surgery will proceed to the sixth surgical state 207 with a probability P(E), and that the surgery will proceed to the seventh surgical state 208 with a probability 1-P(E). Regardless of the choice made, the surgery proceeds to an eighth surgical state 209, in which the spilled bile is suctioned, and then advances to the second surgical state 203.

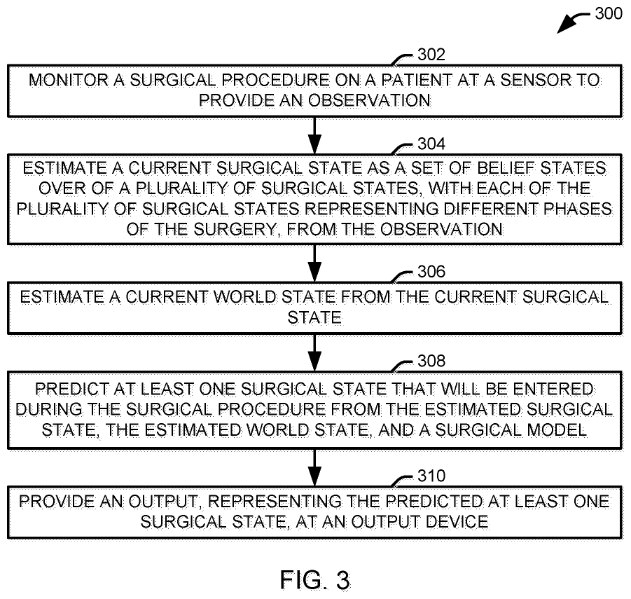

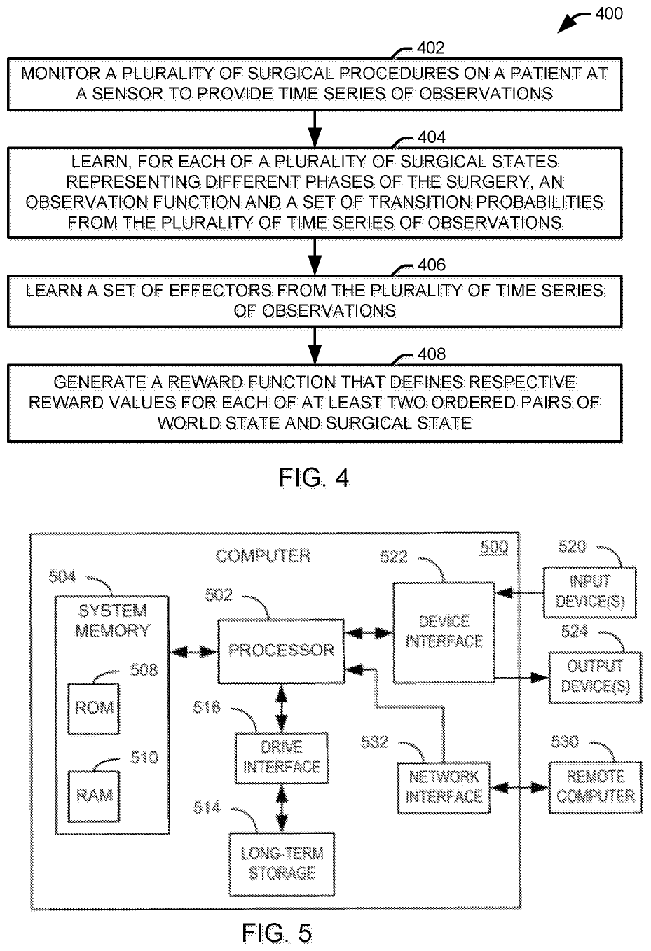

In view of the foregoing structural and functional features described above, methods in accordance with various aspects of the invention will be better appreciated with reference to FIGS. 3 and 4. While, for purposes of simplicity of explanation, the methods of FIGS. 3 and 4 are shown and described as executing serially, it is to be understood and appreciated that the invention is not limited by the illustrated order, as some aspects could, in accordance with the invention, occur in different orders and/or concurrently with other aspects from that shown and described herein. Moreover, not all illustrated features may be required to implement a method in accordance with an aspect of the invention. The example methods of FIGS. 3 and 4 can be implemented as machine-readable instructions that can be stored in a non-transitory computer readable medium, such as can be computer program product or other form of memory storage. The computer readable instructions corresponding to the methods of FIGS. 3 and 4 can also be accessed from memory and be executed by a processing resource (e.g., one or more processor cores).

FIG. 3 illustrates a method 300 for assisting surgical decision making using a model trained via reinforcement learning. It will be appreciated that the method will be implemented by an electronic system, which can include any of dedicated hardware, machine executable instructions stored on a non-transitory computer readable medium and executed by an associated processor, or a combination of these. In practice, the model used by the method will have already been trained on sensor data from a set of previously performed surgical procedures via a supervised or semi-supervised learning process.

At 302, a surgical procedure on a patient is monitored at a sensor to provide an observation. In practice, the sensor can include video cameras, accelerometers disposed on a surgeon or an instrument associated with the surgical procedure, intraoperative imaging technologies, and sensors that detect biometric parameters of the patient. In the illustrated example, the sensor is a surgical vision system. In the illustrated example, the observation from the surgical vision system is obtained by providing the camera output to a discriminative pattern recognition classifier, such as a support vector machine or an artificial neural network, and utilizing an output of the pattern recognition system as the observation. Accordingly, the visual model for the model can be altered based upon a choice of the interpreting pattern recognition system or systems.

At 304, a current surgical state is estimated as a belief state defining probabilities for each of a plurality of surgical states, with each of the plurality of surgical states representing different phases of the surgery, from the observation. Each of the plurality of surgical states is represented by an observation function that defines at least one likelihood of a given observation from the sensor interface given the surgical state. At 306, a world state of a plurality of world states is estimated from the current surgical state and the observation. Each of the plurality of world states represents a state of either the patient or the environment in which the surgical procedure is being conducted. In one implementation, the state estimations at 304 and 306 can be performed by sampling over the set of surgical states and the set of world states and updating the optimal policies, in a manner similar to the randomized variant of a value learning algorithm for partially observable Markov decision processes.

At 308, at least one surgical state that will be entered during the surgical procedure is predicted from the estimated surgical state, the estimated world state, and a surgical model. In one implementation, the model is explored by an agent that models the decisions of a surgeon performing the surgical procedure to determine a series of actions, from a plurality of actions that can be taken by the surgeon to transition between states of the plurality of surgical states. The agent models the decisions of the surgeon under the assumption that the surgeon has full knowledge of the current surgical state, but only partial knowledge of the current world state. In one implementation, the predicted state or states can be used to estimate a likelihood that a resource that will be required for the patient during the surgical procedure.

At 310, an output, representing the predicted at least one surgical state, is provided at an output device. The output can include, for example, a predicted outcome of the surgery, for example, in the form of a surgical state or world state that is expected to be entered during the procedure given the current surgical state and world state, a recommended action for the surgeon, intended to provide a greatest reward for the surgical procedure given the model, a request for a specific resource, such as imaging equipment or scheduled time in an operating room, to a user at an institution associated with the surgical procedure.

FIG. 4 illustrates a method 400 for providing a surgical model, for example, for an assisted surgical decision making method like that presented in FIG. 3. The method of FIG. 4 allows for the model to be formed from methods used and results obtained from the results of a plurality of previous surgeries. At 402, a plurality of surgical procedures are monitored at a sensor to provide a plurality of time series of observations. At 404, for each of a plurality of surgical states representing different phases of the surgery, an observation function and a set of transition probabilities are learned from the plurality of time series of observations. The observation function represents at least one likelihood of a given observation from the sensor given the surgical state. The set of transition probabilities each represent a likelihood of a transition from a given surgical state to another surgical state given each of a specific world state of a plurality of world states and a selected action of a plurality of actions.

In one example, where the sensor is a video camera, the observations are generated via a visual model, implemented as a discriminative classifier model that interprets the visual data. This interpretation can be indirect, for example, by finding objects within the scene that are associated with specific surgical states or world states, or by directly determining a surgical state or world state via the classification process. In one example, the visual model is implemented as an artificial neural network, such as a convolutional neural network, a cluster network, or a recurrent neural network, that is trained on the plurality of time series of observations to identify the surgical state. Since the system is intended to learn from a limited amount of data and under small computational resource, a feature space for generating observations is selected to be concise and representative, with a balance between invariance and expressiveness.

In another implementation, the classification is performed from several visual cues in the videos, categorized broadly as local and global descriptor and motivated by the way surgeons deduce the stage of the surgery. These cues are used to define a feature space that captures the principal axes of variability and other discriminant factors that determine the surgical state, and then the discriminative classifier can be trained on a set of features comprising the defined feature space.

The cues include color-oriented visual cues generated from a training image database of positive and negative images. Other descriptor categories for individual RGB/HSV channels can be utilized to increase dimensionality to discern features that depend on color in combination with some other property. Pixel values can also be used as features directly. The RGB/HSV components can augment both local descriptors (e.g., color values) and global descriptors (e.g., a color histogram).The relative position of organs and instruments is also an important visual cue. The position of keypoints generated via speeded-up robust features (SURF) process can be encoded with an 8×8 grid sampling of a Gaussian surface centered around the keypoint. The variance of the Gaussian defines the spatial “area of influence” of a keypoint. Shape is important for detecting instruments, which can be used as visual cues for identifying the surgical state, although differing instrument preferences among surgeons can limit the value of shape-based cues. Shape can be encoded with various techniques, such as the Viola-Jones object detection framework, using image segmentation to isolate the instruments and match against artificial 3D models, and other methods. For local frame descriptors, a standard SURF descriptor can be used as a base, and for a global frame descriptor, grid-sampled histogram of ordered gradients (HOG) descriptors and discrete cosign transform (DCT) coefficients can be added. Texture is a visual cue used to distinguish vital organs, which tend to exhibit a narrow variety of color. Texture can be extracted using a co-occurrence matrix with Haralick descriptors, by a sampling of representative patches to be evaluated with a visual descriptor vector for each patch, and other methods. In the illustrated example, a Segmentation-based Fractal Texture Analysis (SFTA) texture descriptor is used.

Finally, the augmented descriptors are combined into a single fixed-dimension frame descriptor. For this, a bag of words (BOW) model can be used to standardize the dimensionality of features. A representative vector quantization (VQ) is computed by sampling frames using only local descriptors. Any set of local descriptors can then be represented as a histogram of projections in the fixed VQ dimension. The final combined frame descriptor is then composed of the BOW histogram and the additional dimensions of the global descriptor. In one implementation, the features comprising the final combined frame descriptor can be reduced to a significantly lower dimensional set of data, represented as a coreset that approximates the data in a manner that captures the classification results that would be obtained on the full dataset. One method for generating a coreset for this purpose can be found in U.S. Pat. No. 9,286,312 to Rus et al., issued Mar. 15, 2016, which is hereby incorporated by reference. One example implementation for training the visual model used to generate observations can be found in Machine Learning and Coresets for Automated Real-Time Video Segmentation of Laparoscopic and Robot-Assisted Surgery by Volkov et al. from the 2017 IEEE International Conference on Robotics and Automation, which is also incorporated herein by reference.

This learning process can be supervised or semi-supervised. In one example, each of the time series of observations can be labeled by a human expert with relevant information, such as a current surgical state or world state. In another example, labelling of some of the time series of observations enables training of pattern recognition and agent models so as to allow either prioritization of labeling examples to an expert (active learning) or automatic training with the assumed labeled (semi-supervised learning). Once the observations are labeled with the surgical states, the observation functions for each state can be readily determined as the conditional probabilities that an observation will be received given each state from the labeled observation data. The transition probabilities and corresponding actions can be determined by sampling across the surgical states and the world states in a manner similar to the Baum-Welch algorithm for Markov decision processes.

At 406, a set of effectors is learned from the plurality of time series of observations. Each effector represents a likelihood of a transition between a given world state of the plurality of world states and another world state of the plurality of world states given a specific surgical state. In one example, the effectors are learned by sampling possible latent surgery and patient states, using stochastic gradient ascent. At 408, an associated rewards function is generated that defining respective reward values for each of at least two ordered pairs of world state and surgical state. These reward values can be learned from the time series of observations, for example, by using the patient outcomes for each surgical procedure, or assigned by a human expert based on domain knowledge.

The combination of the discriminative visual model with a generative model for the surgical states gracefully handles large amounts of surgery video data both in the training phase and in online processing of newly incoming video streams and classification of surgical states. The results of the initial classification are used to train a generative model that represents the various surgical states, world states, and the transitions among these states via reinforcement learning. This allows both for efficient training of the generative model as well as real-time decision making by the agent in response to observations provided by the visual model. In one implementation, each of the surgical states and world states are selected by a human expert, such as a surgeon with experience in a given procedure. Alternatively, one or more states can be defined during the training process itself. For example, states can be added or removed via Bayesian non-parametric methods based upon the training data.

FIG. 5 illustrates a computer system 500 that can be employed to implement systems and methods described herein, such as based on computer executable instructions running on the computer system. The computer system 500 can be implemented on one or more general purpose networked computer systems, embedded computer systems, routers, switches, server devices, client devices, various intermediate devices/nodes and/or stand alone computer systems.

The computer system 500 includes a processor 502 and a system memory 504. Dual microprocessors and other multi-processor architectures can also be utilized as the processor 502. For example, GPU and general-purpose GPU system can be used for efficient sampling of possible trajectories and belief states, or network forward and backward computations, at either training or online running time, as these operations are highly parallelizable. The processor 502 and system memory 504 can be coupled by any of several types of bus structures, including a memory bus or memory controller, a peripheral bus, and a local bus using any of a variety of bus architectures. The system memory 504 includes read only memory (ROM) 506 and random access memory (RAM) 508. A basic input/output system (BIOS) can reside in the ROM 506, generally containing the basic routines that help to transfer information between elements within the computer system 500, such as a reset or power-up.

The computer system 500 can include one or more types of long-term data storage 510, including a hard disk drive, a magnetic disk drive, (e.g., to read from or write to a removable disk), and an optical disk drive, (e.g., for reading a CD-ROM or DVD disk or to read from or write to other optical media). The long-term data storage 510 can be connected to the processor 502 by a drive interface 512. The long-term data storage 510 components provide nonvolatile storage of data, data structures, and computer-executable instructions for the computer system 500. A number of program modules may also be stored in one or more of the drives as well as in the RAM 508, including an operating system, one or more application programs, other program modules, and program data.

A user may enter commands and information into the computer system 500 through one or more input devices 522, such as a keyboard or a pointing device (e.g., a mouse). These and other input devices are often connected to the processor 502 through a device interface 524. For example, the input devices can be connected to the system bus by one or more a parallel port, a serial port or a universal serial bus (USB). One or more output device(s) 526, such as a visual display device or printer, can also be connected to the processor 502 via the device interface 524.

The computer system 500 may operate in a networked environment using logical connections (e.g., a local area network (LAN) or wide area network (WAN) to one or more remote computers 530. A given remote computer 530 may be a workstation, a computer system, a router, a peer device or other common network node, and typically includes many or all of the elements described relative to the computer system 500. The computer system 500 can communicate with the remote computers 530 via a network interface 532, such as a wired or wireless network interface card or modem. In a networked environment, application programs and program data depicted relative to the computer system 500, or portions thereof, may be stored in memory associated with the remote computers 530.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A system comprising:
at least one sensor positioned to monitor a surgical procedure on a patient;
a processor; and
a non-transitory computer, storing machine executable instructions for providing a surgical decision support system, the machine executable instructions being executed by the processor to provide:
a sensor interface that receives data from the at least one sensor and generates observations from the received data;
a surgical model, comprising:
a plurality of surgical states, each representing different phases of the surgery;
an observation function for each surgical state representing at least one likelihood of a given observation from the sensor interface given the surgical state;
a plurality of actions that can be taken by a surgeon to transition between states of the plurality of surgical states;
a plurality of world states, each representing a state of one of the patient, a set of medical professionals performing the surgical procedure, the set of medical processionals including the surgeon, and the environment in which the surgical procedure is being conducted;
a set of effectors, each representing a likelihood of a transition between a given world state and another world state given a specific surgical state;
a set of transition probabilities, each representing a likelihood of a transition from a given surgical state to another surgical state given each of a specific world state and an selected action of the plurality of actions; and
a rewards function defining respective reward values for each of at least two ordered pairs, each of the at least two ordered pairs representing a surgical state of the plurality of surgical states and a world state of the plurality of world states;
an agent that estimates a current surgical state and a current world state as a belief state from at least one observation from the sensor interface and selects at least one of the plurality of actions as to optimize an expected reward given the belief state; and
a user interface that provides one of the selected at least one of the plurality of actions, a likelihood that a selected surgical state will be entered in the course of the procedure, and an expected final world state to an associated output device; and
the output device, which provides the one of the selected at least one of the plurality of actions, the likelihood that the selected surgical state will be entered in the course of the procedure, and an expected final world state to a user in a form comprehensible by a human being.

2. The system of claim 1, wherein the at least one sensor comprises a camera that captures frame of video and the sensor interface comprises a pattern recognition classifier configured to identify objects in the frames of video.

3. The system of claim 2, wherein the pattern recognition classifier is one of a support vector machine, recurrent neural network, a convolutional neural network, and a capsule network.

4. The system of claim 1, wherein the output device comprises a network interface that, in response to the likelihood that the selected surgical state will be entered exceeding a threshold value, transmits a request to a device associated with a member of a surgical team for the surgical procedure instructing the member to prepare an additional resource for the surgical procedure.

5. The system of claim 1, wherein the output device provides an alert to the surgeon to advise a change in a surgical plan associated with the surgical procedure in response to the expected final world state.

6. The system of claim 1, wherein the agent selects at least one action by modeling the decisions of the surgeon to determine a series of the plurality of actions under the assumption that the surgeon has full knowledge of a current surgical state, but only partial knowledge of a current world state.

7. A method comprising:
monitoring a surgical procedure on a patient at a sensor to provide an observation;
estimating a current surgical state as a belief state defining probabilities for each of a plurality of surgical states, each of the plurality of surgical states representing different phases of the surgery, from the observation and an observation function for each of the plurality of surgical states representing at least one likelihood of a given observation from the sensor interface given the surgical state;
estimating a world state of a plurality of world states from the current surgical state and the observation, each of the plurality of world states representing a state of one of the patient and the environment in which the surgical procedure is being conducted;
predicting, from the estimated surgical state, the estimated world state, and a model, at least one surgical state that will be entered during the surgical procedure;
and providing an output, at an associated output device, representing the predicted at least one surgical state.

8. The method of claim 7, further comprising estimating a likelihood that a given resource that will be required for the patient from the predicted at least one surgical state, wherein providing the output comprises transmitting a request for the given resource to a user at an institution associated with the surgical procedure.

9. The method of claim 7, wherein providing the output comprises communicating a recommended action to the surgeon given the predicted at least one surgical state.

10. The method of claim 7, wherein monitoring the surgical procedure on a patient at the sensor to provide the observation comprises providing data from the sensor to a discriminative pattern recognition classifier to provide the observation as a classification output.

11. The method of claim 7, wherein at least one of the plurality of world states represents a physical condition of the patient.

12. The method of claim 7, wherein at least one of the plurality of world states represents an attribute of a medical professional performing the surgical procedure.

13. The method of claim 7, further comprising providing the model, wherein providing the model comprises:
monitoring a plurality of surgical procedures to provide a plurality of time series of observations;
annotating each of the plurality of time series such that each observation is associated with a corresponding surgical state and a set of world states;
learning each of a set of transition probabilities, each representing a likelihood of a transition from a given surgical state to another surgical state given each of a specific world state and an selected action of a plurality of actions, a set of effectors, each representing a likelihood of a transition between a given world state and another world state given a specific surgical state, and an observation function for each of the plurality of surgical states representing at least one likelihood of a given observation from the sensor interface given the surgical state from the annotated plurality of time series; and
generating an associated rewards function defining respective reward values for each of at least two ordered pairs, each of the at least two ordered pairs representing a surgical state of the plurality of surgical states and a world state of the plurality of world states.

14. A method for providing a model, comprising:
monitoring a plurality of surgical procedures at a sensor to provide a plurality of time series of observations;
learning, for each of a plurality of surgical states representing different phases of a surgical procedure, an observation function representing at least one likelihood of a given observation from the sensor given the surgical state and a set of transition probabilities, each representing a likelihood of a transition from a given surgical state to another surgical state given each of a specific world state of a plurality of world states and a selected action of a plurality of actions, from the plurality of time series of observations;
learning a set of effectors, each representing a likelihood of a transition between a given world state of the plurality of world states and another world state of the plurality of world states given a specific surgical state, from the plurality of time series of observations; and
generating an associated rewards function defining respective reward values for each of at least two ordered pairs, each of the at least two ordered pairs representing a surgical state of the plurality of surgical states and a world state of the plurality of world states.

15. The method of claim 14, further comprising:
monitoring a surgical procedure on a patient at the sensor to provide an observation;
estimating a current surgical state as a belief state defining probabilities for each of a plurality of surgical states from the observation and an observation function for each of the plurality of surgical states representing at least one likelihood of a given observation from the sensor interface given the surgical state;
estimating a world state of a plurality of world states from the current surgical state;
predicting, from the estimated surgical state, the estimated world state, and from a model, at least one surgical state that will be entered during the surgical procedure; and
providing an output, at an associated output device, representing the predicted at least one surgical state.

16. The method of claim 14, further comprising annotating each of the plurality of time series such that each observation is associated with a corresponding surgical state of the plurality of surgical states and a world state of the plurality of world states.

17. The method of claim 16, wherein annotating each of the plurality of time series comprises providing data from the sensor to a pattern recognition system.

18. The method of claim 14, wherein each of the plurality of surgical states, and the plurality of world states are selected by a human expert.

19. The method of claim 14, wherein at least one of the plurality of surgical states are generated by an expert system from the plurality of time series of observations.

20. The method of claim 14, wherein the sensor is a camera and each of the time series of observations comprises a video of a surgical procedure of the plurality of surgical procedures.
